(19) United States
(12) Patent Application Publication
(10) Pub. No.: US 2025/0284763 A1
(43) Pub. Date: Sep. 11, 2025

(54) GENERATIVE SCHRÖDINGER NETWORK

(71) Applicant: International Business Machines Corporation, Armonk, NY (US)

(72) Inventors: Thanh Lam Hoang, Maynooth (IE); Marcos Martínez Galindo, Dublin (IE); Lam Minh Nguyen, Ossining, NY (US); Niall Robertson, Dublin (IE); Vanessa Lopez Garcia, Dublin (IE)

(21) Appl. No.: 18/600,976

(22) Filed: Mar. 11, 2024

(51) Int. Cl. G06F 17/13

(52) U.S. Cl. CPC G06F 17/13

(57) ABSTRACT

Embodiments are directed to a computer-implemented method that includes executing a generative network that includes a generative model of a system under evaluation (SUE). The generative model is operable to model a probability density function of the SUE to generate electron coordinates of electrons in the SUE. The electron coordinates are generated by the generative model in a manner that minimizes an estimated energy of the SUE. The electron coordinates generated by the generative model can be sampled in parallel.

### BACKGROUND

The present invention relates generally to programmable computers. More specifically, the present invention relates to computer-implemented methods, programmable computer systems, and computer program products operable to generate and implement a novel generative network, which is referred to herein as a generative Schrödinger network, operable to solve the Schrödinger equation via modelling the relevant wave-function as a generative model.

The Schrödinger equation is a linear partial differential equation that governs the wave function of a quantum-mechanical system. Tasks performed in various domains such as drug discovery, new material discovery, and the like rely on the ability to solve the Schrödinger equation because such solutions can provide insights into discovering, for example, how drugs bind to proteins, the chemical and physical properties of drugs, how defects on materials develop, how new materials withstand different extreme conditions, and the like. Artificial intelligence (AI) techniques have been proposed for solving the Schrödinger equation.

### SUMMARY

Embodiments are directed to a computer-implemented method that includes executing a generative network that includes a generative model of a system under evaluation (SUE). The generative model is operable to model a probability density function of the SUE to generate electron coordinates of electrons in the SUE. The electron coordinates are generated by the generative model in a manner that minimizes an estimated energy of the SUE. The electron coordinates generated by the generative model can be sampled in parallel.

Embodiments of the invention further provide computer systems and a computer program products having substantially the same features and as the above-described computer-implemented method.

Additional features and advantages are realized through the techniques described herein. Other embodiments and aspects are described in detail herein. For a better understanding, refer to the description and to the drawings.

### BRIEF DESCRIPTION OF THE DRAWINGS

The subject matter which is regarded as the present disclosure is particularly pointed out and distinctly claimed in the claims at the conclusion of the specification. The foregoing and other features and advantages are apparent from the following detailed description taken in conjunction with the accompanying drawings in which:

FIG. 1 depicts a computing environment operable to implement aspects of the invention;

FIG. 2 depicts a graph illustrating a plot of the square of a wave function (Ψ²) vs. a distance of an electron from its nucleus in accordance with aspects of the invention;

FIG. 3A depicts equations used in connection with aspects of the invention;

FIG. 3B depicts additional equations used in connection with aspects of the invention;

FIG. 3C depicts additional equations used in connection with aspects of the invention;

FIG. 3D depicts additional equations used in connection with aspects of the invention;

FIG. 4A depicts additional details of an equation used in connection with aspects of the invention;

FIG. 4B depicts electrons and nuclei of a system-under-evaluation (SUE) operable to be evaluated using aspects of the invention;

FIG. 5A depicts additional details of an equation used in connection with aspects of the invention;

FIG. 5B depicts electrons and nuclei of a system-under-evaluation (SUE) operable to be evaluated using aspects of the invention;

FIG. 6A depicts additional details of an equation used in connection with aspects of the invention;

FIG. 6B depicts electrons and nuclei of a system-under-evaluation (SUE) operable to be evaluated using aspects of the invention;

FIG. 7 depicts a plot illustrating a Markov Chain Monte Carlo (MCMC) sampling method;

FIG. 8 depicts a system embodying aspects of the invention;

FIG. 9 depicts a generative network that can be used to implement aspects of the invention.

FIG. 10 depicts a computer-implemented method in accordance with aspects of the invention;

FIG. 11 depicts an algorithm operable to implement the computer-implemented method shown in FIG. 10;

FIG. 12 depicts a system embodying aspects of the invention;

FIG. 13A depicts a univariate Gaussian distribution in accordance with aspects of the invention;

FIG. 13B depicts a multivariate Gaussian distribution in accordance with aspects of the invention;

FIG. 14 depicts test results illustrating performance of a system in accordance with aspects of the invention; and

FIG. 15 depicts test results illustrating performance of a system in accordance with aspects of the invention.

In the accompanying figures and following detailed description of the disclosed embodiments, the various elements illustrated in the figures are provided with three or four digit reference numbers. The leftmost digit(s) of each reference number corresponds to the figure in which its element is first illustrated.

### DETAILED DESCRIPTION

Embodiments are directed to a computer-implemented method that includes executing a generative network that includes a generative model of a system under evaluation (SUE). The generative model is operable to model a probability density function of the SUE to generate electron coordinates of electrons in the SUE. The electron coordinates are generated by the generative model in a manner that minimizes an estimated energy of the SUE. The electron coordinates generated by the generative model can be sampled in parallel.

The above-described embodiments of the invention provide technical benefits and technical effects. For example, the above-described computer-implemented method introduces an approach for solving the Schrödinger equation. Different from existing machine learning work presenting wave functions as neural networks, the computer-implemented method directly models the generative process as a neural network from which an approximation form of the wave function is derived. The benefit of using a generative model is that the electron coordinates of the SUE can be sampled from the generative models in parallel and thus it is more efficient than sequential sampling approaches such as Markov Chain Monte Carlo (MCMC) methods or alternative methods like diffusion Monte Carlo for estimating the integral involved in the energy calculation.

In addition to any one or more of the features described herein, the estimated energy of the SUE is associated with multiple energy sources.

The above-described embodiments of the invention provide technical benefits and technical effects. For example, the novel generative network estimates the integral using the random samples from the generative networks in parallel. Thus, the generative network is more efficient than known approaches (e.g., neural networks embodied in VMC, MCMC, and the like) to applying AI techniques to solve the Schrödinger equation. The generative networks further facilitate the effective and efficient capture of multiple energy sources reflected in the Schrödinger equation.

In addition to any one or more of the features described herein, the multiple energy sources include a Laplacian energy source, an electron-nuclei energy source, and an electron-electron energy source.

The above-described embodiments of the invention provide technical benefits and technical effects. For example, the novel generative network estimates the integral using the random samples from the generative networks in parallel. Thus, the generative network is more efficient than known approaches (e.g., neural networks embodied in VMC, MCMC, and the like) to applying AI techniques to solve the Schrödinger equation. The generative networks further facilitate the effective and efficient capture of multiple energy sources reflected in the Schrödinger equation, including but not limited to a Laplacian energy source, an electron-nuclei energy source, and an electron-electron energy source.

In addition to any one or more of the features described herein, the probability density function includes a multivariate Gaussian distribution.

The above-described embodiments of the invention provide technical benefits and technical effects. For example, embodiments of the invention using the disclosed generative network can include any form of distribution that has a closed-form density function, including a multivariate Gaussian distribution.

In addition to any one or more of the features described herein, the computer-implemented further includes using an anti-symmetry correction model to apply an anti-symmetry correction function to the probability density function generated by the generative model.

The above-described embodiments of the invention provide technical benefits and technical effects. For example, embodiments of the invention uses the above-described generative network to also takes into account anti-symmetry considerations. Because electrons are fermions, they must follow the Pauli-exclusion principle, i.e., the wave functions of same-spin electrons must be anti-symmetric. Specifically, the sign of the function must be changed when two random coordinates of the same-spin electrons are exchanged. The generative network provides a method that regularizes the wave function such that it has a shape similar to an anti-symmetric function

In addition to any one or more of the features described herein, the computer-implemented method further includes combining an output of the probability density function generated by the generative model and the anti-symmetry correction function.

The above-described embodiments of the invention provide technical benefits and technical effects. For example, a random coordinate samples module can be used to draw K random samples from the mixture of Gaussian components in a generative model. An anti-symmetry correction model can be used to get the value of the anti-symmetric correction function at the sampled coordinates. An anti-symmetry constraint module is used to get the estimated value of the system energy using the random sampled coordinates. The anti-symmetry constraint module is used to get the loss with respect to the anti-symmetry constraint. A loss minimization module is used to gain the overall loss as the sum of the energy and the antisymmetric constraints. A loss minimization module is used to perform gradient updates when optimizing the energy and the antisymmetric constraint alternatively.

In addition to any one or more of the features described herein, combining the output of the probability density function generated by the generative model and the anti-symmetry correction function creates an anti-symmetry constraint loss and an estimate of an energy of the SUE.

The above-described embodiments of the invention provide technical benefits and technical effects. For example, a random coordinate samples module can be used to draw K random samples from the mixture of Gaussian components in a generative model. An anti-symmetry correction model can be used to get the value of the anti-symmetric correction function at the sampled coordinates. An anti-symmetry constraint module is used to get the estimated value of the system energy using the random sampled coordinates. The anti-symmetry constraint module is used to get the loss with respect to the anti-symmetry constraint. A loss minimization module is used to gain the overall loss as the sum of the energy and the antisymmetric constraints. A loss minimization module is used to perform gradient updates when optimizing the energy and the antisymmetric constraint alternatively.

Accordingly, the above-described embodiments of the disclosure provide a system and method that takes input as an electronic system with atoms and atomic numbers; automatically creates a generative model with a closed form of density function (this is used to calculate the energy of the system) to approximate the wavefunction of the input system; and sample the electron coordinates randomly from the given generative model in parallel efficiently. The generative model is trained to minimize the estimated energy of the system and the solution of the Schrödinger equation is given by the trained generative model. Antisymmetric correction is deployed via ingesting into the loss of training the generative models to optimize the similarity between the generative model density and the corrective antisymmetric function besides minimizing the energy.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

Many of the functional units described in this specification are illustrated as logical blocks such as modules, processors, and the like. Embodiments of the invention apply to a wide variety of implementations of the logical blocks described herein. For example, a given logical block can be implemented as a hardware circuit operable to include custom VLSI circuits or gate arrays, as well as off-the-shelf semiconductors such as logic chips, transistors, or other discrete components. The logical blocks can also be implemented in programmable hardware devices such as field programmable gate arrays, programmable array logic, programmable logic devices, and the like. The logical blocks can also be implemented in software for execution by various types of processors. Some logical blocks described herein can be implemented as one or more physical or logical blocks of computer instructions which can, for instance, be organized as an object, procedure, or function. The executables of a logical block described herein need not be physically located together but can include disparate instructions stored in different locations which, when joined logically together, include the logical block and achieve the stated purpose for the logical block.

The various components/modules of the systems illustrated herein are depicted separately for ease of illustration and explanation. In embodiments of the invention, the functions performed by the various components/modules can be distributed differently than shown without departing from the scope of the various embodiments of the invention describe herein unless it is specifically stated otherwise.

Turning now to an overview of technologies that are more specifically related to aspects of the invention, classical physics, which is the collection of theories that existed before the advent of quantum mechanics, describes many aspects of nature at an ordinary (macroscopic) scale. However, classical physics is not sufficient for describing aspects of nature at small (atomic and subatomic) scales. Most theories in classical physics can be derived from quantum mechanics as an approximation valid at large (macroscopic) scale. Unlike systems based on classical physics, systems based on quantum mechanics (i.e., quantum systems) have bound states quantized to discrete values of energy, momentum, angular momentum, and other quantities; measurements of quantum systems show characteristics of both particles and waves (wave-particle duality); and there are limits to how accurately the value of a physical quantity can be predicted prior to its measurement, given a complete set of initial conditions (the uncertainty principle).

The Schrödinger equation is a linear partial differential equation that describes the behavior or change of quantum systems. Known machine-learning-based and deep-learning-based approaches to solving the Schrödinger equation approximate the Schrödinger equation by searching for the optimal neural networks representing the wave functions that minimize the system energy. However, because the exact energy calculation requires integral evaluations, known machine-learning-based and deep-learning-based approaches to solving the Schrödinger equation rely on MCMC methods or alternative methods like diffusion Monte Carlo for estimating the integral involved in the energy calculation. Known machine-learning-based and deep-learning-based approaches to solving the Schrödinger equation sample the energies at multiple points to teach themselves to search for the molecule's ground state.

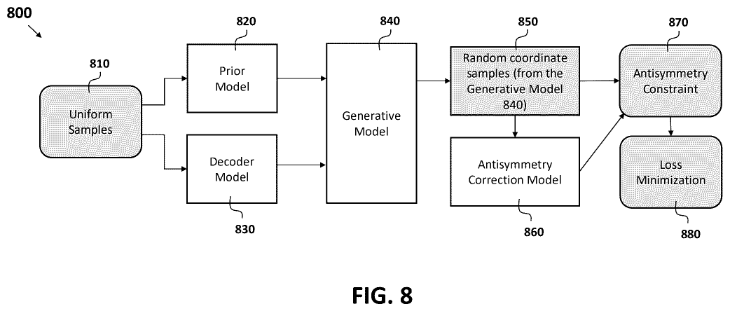

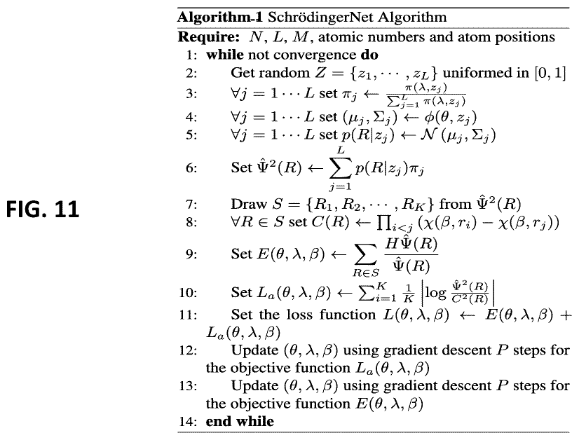

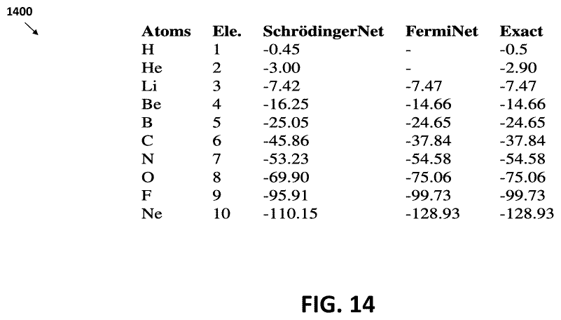

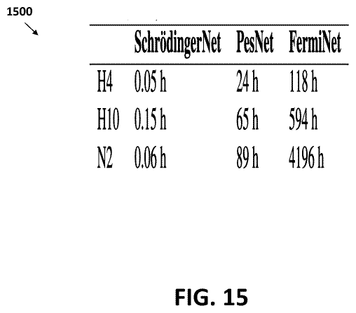

Embodiments of the invention introduce a new method that relies on generative models for generating random samples from a target distribution. Different from methods that rely on MCMC-based methods, embodiments of the invention are more efficient in that they rely on generative models configured to generate an arbitrarily large set of unbiased random samples in parallel. Using the disclosed generative Schrödinger network (e.g., the generative Schrödinger network 800, 800A shown in FIGS. 8 and 11), learning to solve the Schrödinger equation is turned into searching for generative models such that its coupled wave function minimizes the energy of the system rather than searching for the optimal wave function directly. Moreover, because the generative models used in embodiments of the invention generate electron coordinates as a function of its parameters, all three parts of the local energy, including the Laplacian, the electron-nuclei, and the electron-electron potential energies contribute to the gradient computing operations performed during the gradient-based learning updates. The performance of the generative Schrödinger network 800 disclosed herein was tested, and the test results demonstrate that the method used in the generative Schrödinger network 800 can approximate the wave function of a system with known ground-state energy with reasonable accuracy (shown in FIG. 14), while it is also able to scale to very large systems with efficient solving time (shown in FIG. 15).

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

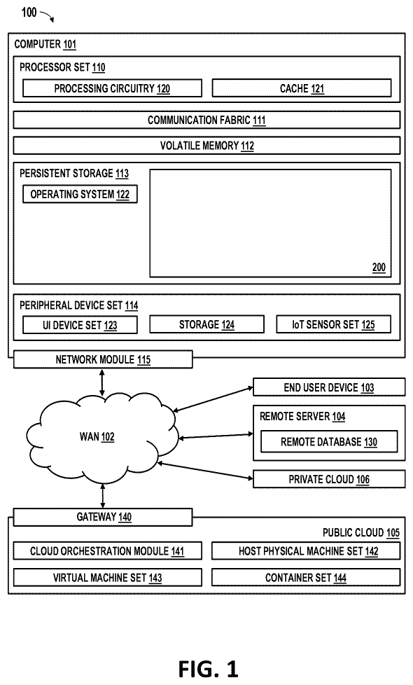

FIG. 1 depicts a computing environment 100 that contains an example of an environment for the execution of at least some of the computer code involved in performing the inventive methods, such as code block 200 operable to implement a novel generative network referred to herein as a generative Schrödinger network having the features and functionality described herein. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

Turning now to a more detailed description of aspects of the present invention, information about the Schrödinger equation utilized in embodiments of the invention will now be provided. Fundamental particles, such as electrons, can be described as particles or waves. Electrons can be described using a wave function. The wave function's symbol is the Greek letter psi, Ψ. The wave function Ψ is a mathematical expression that carries information about the electron it is associated with. From the wave function, it is possible to obtain the electron's energy, angular momentum, and orbital orientation. The wave function can have a positive or negative sign. Wave functions with like signs (waves in phase) will interfere constructively, leading to the possibility of bonding. Wave functions with unalike signs (waves out of phase) will interfere destructively.

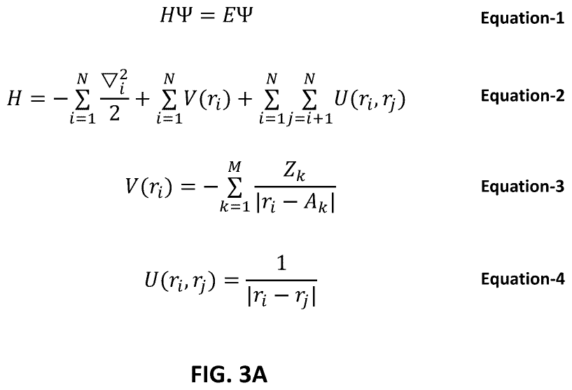

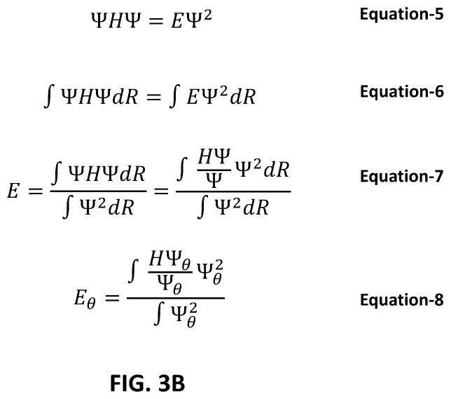

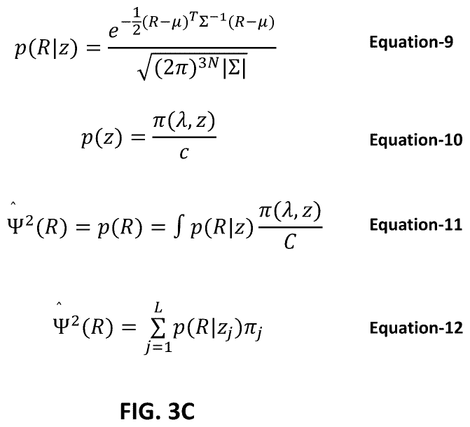

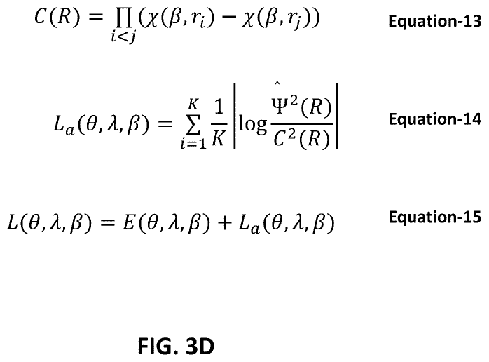

In 1926, Erwin Schrödinger deduced the wave function for the simplest of all atoms, hydrogen, which resulted in the Schrödinger equation (Equation-1 shown in FIG. 3A). Solving the Schrödinger equation enables scientists to determine wave functions for electrons in atoms and molecules. The Schrödinger equation is an equation of quantum mechanics, which means that calculated wave functions have discrete, allowed values for electrons bound in atoms and molecules, and all other values are forbidden.

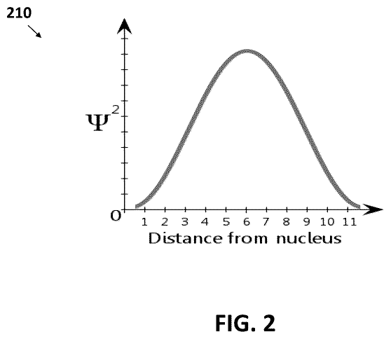

In addition to the importance of Ψ, its square Ψ² also has enormous significance in chemistry. Ψ² is the probability density and can be used to identify where an electron is most likely to be found in the space around its associated nucleus. For example, in the (fictitious) schematic diagram plotted on the plot 210 shown in FIG. 2, Ψ² is plotted against a distance of the electron from its associated. It can be seen from the plot 210 that the electron is most likely to be found between about 5-7 units from the nucleus. It can also be seen that there is a vanishingly small likelihood that the electron will be at the nucleus more than about 11½ units away from the nucleus. There is a 100 percent probability that the electron is somewhere—in other words a probability of 1.

As previously noted, the Schrödinger equation is a linear partial differential equation that governs the wave function of a quantum-mechanical system. Tasks performed in various domains such as drug discovery, new material discovery, and the like rely on the ability to solve the Schrödinger equation because it can provide insights into discovering, for example, how drugs bind to proteins, the chemical and physical properties of drugs, how defects on materials develop, how new materials withstand different extreme conditions, and the like.

Embodiments of the invention provide programmable computer systems, computer-implemented methods, and computer program products operable to generate and implement a novel generative network, which is referred to herein as a generative Schrödinger network (e.g., systems 800, 800A shown in FIG. 8 and FIG. 11, respectively) operable to solve the Schrödinger equation via modelling the relevant wave-function as a generative model (e.g., generative model 840, 840A shown in FIG. 8 and FIG. 11, respectively). Generative modeling is a type of unsupervised learning problem that automatically discovers and learns the regularities or patterns in input data in such a way that the model can be used to generate or output new examples that plausibly could have been drawn from the original dataset. The significance of generative models lies in their ability to create, which has vast implications in various fields, from art to science. Generative models can play a pivotal role in tasks that require the creation of new content, examples of which include synthesizing realistic human faces, composing music, or even generating textual content. Examples of unsupervised generative algorithms include generative adversarial networks (GANs) and auto-encoders (AEs) (e.g., a variational AE (VAE)).

The distinction between generative and discriminative models is fundamental in machine learning. Generative models focus on understanding how the data is generated in order to learn the distribution of the data itself. For example, when evaluating pictures of cats and dogs, a generative model would try to understand what makes a cat look like a cat and a dog look like a dog so that the generative model can eventually generate new images that resemble either cats or dogs. On the other hand, discriminative models focus on distinguishing between different types of data. Discriminative models don't necessarily learn or understand how the data is generated. Instead, they learn the boundaries that separate one class of data from another. Using the same example of cats and dogs, a discriminative model would learn to tell the difference between the two, but it wouldn't necessarily be able to generate a new image of a cat or dog on its own.

Returning now to the Schrödinger equation utilized in embodiments of the invention, given a system with N electrons and M nuclei, the Schrödinger equation of the system is defined by Equation-1 shown in FIG. 3A. In Equation-1, Ψ(r₁, r₂, . . . , r_{N}, A₁, A₂, . . . , A_{M}) is a wave function of the N three-dimensional coordinates of electrons and M three-dimensional coordinates of nuclei, E is the system energy, and H is a Hamiltonian operator. The Born-Oppenheimer approximation assumes that the electronic motion and the nuclear motion in molecules can be separated because nuclei are much heavier and slower than electrons, so their coordinates A₁, A₂, . . . , A_{M} can be considered as constants relative to the coordinates of smaller but faster electrons. Therefore, the wave function can be simplified to a function of electron coordinates Ψ(r₁, r₂, . . . , r_{N}), and solutions to the Schrödinger equation HΨ=EΨ (Equation-1 shown in FIG. 3A) are the wave-functions of electron coordinates Ψ(r₁, r₂, . . . , r_{N}). The probabilistic meaning of the wave-function is given by Ψ²(r₁, r₂, . . . , r_{N}), which provides the probability distribution that indicates where electrons under evaluation can be found in the 3D space. For simplicity, the coordinates of the electrons in the systems are denoted as R=(r₁, r₂, . . . , r_{N}).

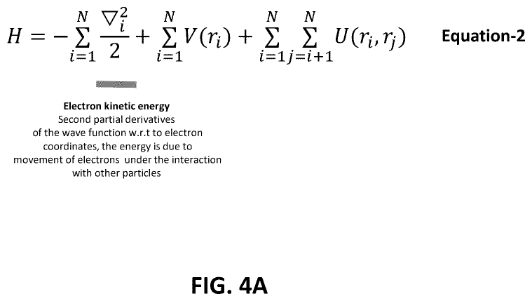

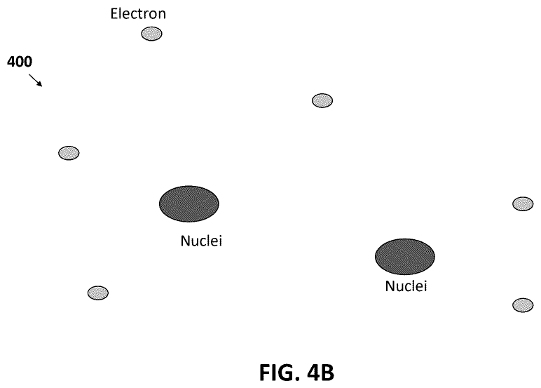

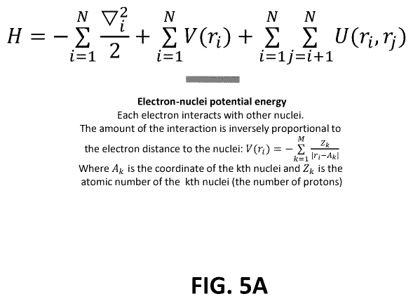

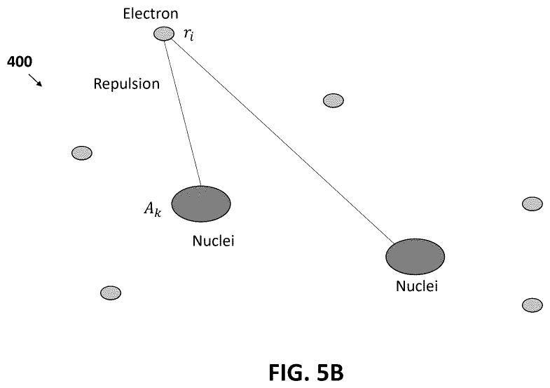

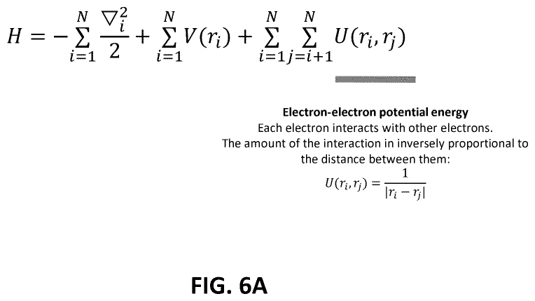

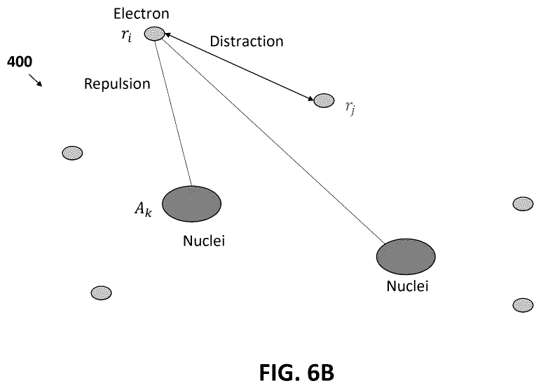

In a Coulomb system, under the atomic unit, the Hamiltonian operator (H) is defined by Equation-2 shown in FIG. 3A and FIG. 4A. In quantum mechanics, the Hamiltonian of a system is an operator corresponding to the total energy of that system, including both kinetic energy and potential energy. Its spectrum, the system's energy spectrum or its set of energy eigenvalues is the set of possible outcomes obtainable from a measurement of the system's total energy. FIG. 4B illustrates an example system-under-evaluation (SUE) 400 having two (2) Nuclei and six (6) Electrons. In general, the Electrons and the Nuclei interact with one another. Because the Electrons have negative charges, they interact with one another by trying to push each other away. Because the Electrons have negative charges and the Nuclei have positive charges, they interact with one another by trying to bring each other closer together. Thus, it can be seen that in the SUE 400, there are multiple forces at play, moving the Electrons and the Nuclei around, which results in different types of energy. These different types of energy (or energy sources) are reflected in the Hamiltonian operator (H) (shown in FIGS. 3A and 4A). For example, as illustrated in FIG. 4A, the first term in the Hamiltonian operator (H) (Equation-2) is the kinetic energy of the system (i.e., SUE 400), where ∇ᵢ² is a Laplacian operator or the second partial derivative. This kinetic energy reflected by the first term is the energy created by the movement of Electrons in the SUE 400. The second term in Equation-2 corresponds to potential energy V(rᵢ) for each Electron interacting with each Nuclei in the SUE 400, as best shown in FIG. 4B. In a Coulomb system, this second term is defined by Equation-3 (shown in FIGS. 3A and 5A), where Aₖ is the coordinate of the kth nuclei, and Zₖ is the atomic number of the kth nuclei (the number of protons). Finally, the last term in the Hamiltonian operator (H) (Equation-2 shown in FIGS. 3A and 6A) is the interaction between the Electrons in the SUE 400 (shown in FIG. 6B), which is defined by Equation-4 (shown in FIGS. 3A and 6A). In a Coulomb system, the interaction between the Electrons in the SUE 400 (shown in FIG. 6B) is proportional to the distance between the two Electrons rᵢ and rⱼ.

Solving Equation-1 can provide the wave function, which will indicate a location where the electron can be found. For example, in some SUE (e.g., SUE 400), the wave function can be used to discover that one electronic moves around the nuclei more than other electrons. Equation-1 governs a variety of observable phenomenon from drug discover to new material discover because when Equation-1 is solved, insights are provided as to how a drug binds to a protein, or how a material becomes very stable with respect to a condition such as high temperature.

Recent attempts to solve Equation-1 have attempted to apply AI-related techniques. Additional computations are applied to Equation-1 to determine the energy component E of Equation-1 and prepare to apply AI techniques to solve Equation-1. Starting from the Schrödinger equation (e.g., Equation-1 shown in FIG. 3A), both sides of the Schrödinger equation can be multiplied by the wave function variable Ψ, which results in Equation-5 (shown in FIG. 3B). An integral operation is applied to both sides of Equation-5 to arrive at Equation-6, where dR represents the space over the electron coordinates (e.g., as shown by SUE 400 in FIGS. 4B, 5B, 6B), and the integral operation is taken over the space of the electron coordinates (e.g., as shown by SUE 400 in FIGS. 4B, 5B, 6B). Because E is a constant with respect to the electron coordinates dR in Equation-6, E can be isolated by dividing both sides of Equation-6 by ∫Ψ² dR to arrive a formula for calculating the energy E in Equation-1 using Equation-7 (shown in FIG. 3B). In general, the goal when solving Equation-7 is to find the wave function Ψ that minimizes the energy E of the system (e.g., SUE 400) to its ground-state. In general, ground state energy is the minimum energy of the system (e.g., SUE 400), and the wave function Ψ where the system achieves the minimum energy is called the ground-state wave function. However, there are difficulties with solving Equation-7, including that the wave-function variable Ψ is initially unknown, and the integrals of the wave function variable Ψ are difficult to estimate.

As previously noted herein, various artificial AI techniques have been proposed for solving the Schrödinger equation (Equation-1) through finding the wave function Ψ that minimizes the energy E (e.g., Equation-7 shown in FIG. 3B) of the system (e.g., SUE 400) to its ground-state. Examples of such AI techniques include, for example, variational Monte Carlo (VMC) techniques in which the wave function is parameterized by a neural network. In the context of neural networks, parameters refer to the variables that determine the behavior of the network during the learning process. These parameters are numerical values that are learned and adjusted by the network through a process called training or optimization. They represent the internal state of the neural network and influence how it processes and transforms input data to produce desired output. There are two main types of parameters in a neural network, namely, weights and biases. Weights are the numeric values associated with the connections between neurons. Each connection between two neurons has an associated weight, which determines the strength or importance of the connection. During training, the neural network adjusts these weights to optimize the network's performance on a given task. These weights essentially control the contribution of each input feature to the network's decision-making process. Adjusting the weights impacts the network's ability to learn and recognize patterns in the input data. Biases are additional parameters used to adjust the output of individual neurons in a neural network. Each neuron typically has a bias value that is added to the weighted sum of its inputs before applying an activation function. Biases enable the network to become more flexible and capable of learning non-linear relationships between inputs and outputs. They shift the activation function of a neuron and allow the network to better fit complex patterns in the data. The values of these parameters are initialized randomly before training begins. As the neural network receives input data and propagates it forward, the values of the parameters are adjusted using optimization algorithms such as gradient descent. The objective of training is to find the optimal set of weights and biases that minimize the network's loss function, allowing it to make accurate predictions or classifications on new, unseen data.

In VMC methods, the wave function is parametrized by a neural network to Ψ_{θ}(R)=Ψ(θ, R) with the parameter θ. Therefore, the energy function of the parameter θ is given by Equation-8 (shown in FIG. 3B). VMC methods minimize E_{θ} with respect to parameter θ to find the ground-state energy and the optimal wave-function represented as a neural network. In the VMC method, generating an estimation of the energy E_{θ} as shown in Equation-8 is not a trivial task as it involves the integral approximation where the normalization factor ∫Ψ_{θ}² is unknown. Because the exact energy calculation for E_{θ} requires integral evaluations, known machine-learning-based and deep-learning-based approaches to solving the Schrödinger equation (through Equation-1 and Equation-7) rely on the Markov Chain Monte Carlo (MCMC) methods or alternative methods like diffusion Monte Carlo for estimating the integral involved in the energy calculation. Known machine-learning-based and deep-learning-based approaches to solving the Schrödinger equation sample the energies at multiple points to teach themselves to search for the molecule's ground state.

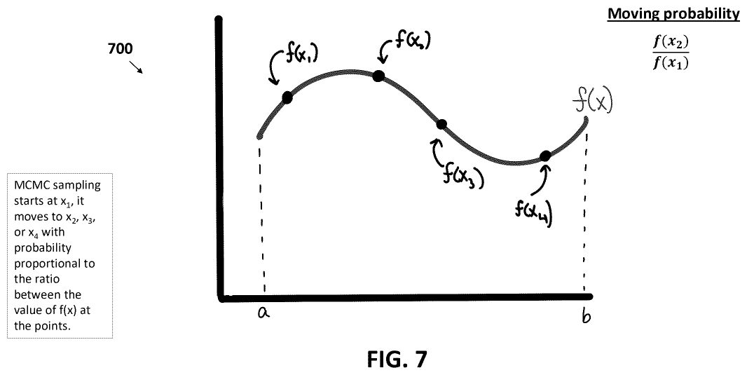

MCMC is a sampling-based method for estimating the integral format of E_{θ} as shown in Equation-8, in which it is not necessary to know the normalization factor ∫Ψ_{θ}² but only Ψ_{θ}². An example of MCMC sampling is shown by a graph 700 in FIG. 7. As shown in FIG. 7, MCMC sampling starts at x₁, and it moves to x₂, x₃, or x₄ with probability proportional to the ratio between the value of f(x) at the points. Thus, MCMC is a sequential sampling approach. Starting with a random point x₁ it moves to a new point x₂ with probability equal to f(x₂)/f(x₁) MCMC is slow because of its sequential nature. The sequential nature of MCMC also makes it difficult to scale to larger data sets.

Embodiments of the invention provide computer-implemented methods, programmable computer systems, and computer program products operable to generate and implement a novel generative network 800, which is referred to herein as a generative Schrödinger network 800. In accordance with aspects of the invention, the generative Schrödinger network 800 is operable to solve the Schrödinger equation (Equation-1 shown in FIG. 3A and Equation-7 shown in FIG. 3B) via modelling the relevant wave-function as a generative model 840. In contrast to known approaches (e.g., neural networks embodied in VMC, MCMC, and the like) to applying AI techniques to solve the Schrödinger equation (e.g., through Equation-1 and Equation-7 shown in FIG. 3A and FIG. 3B, respectively), embodiments of the invention do not model the wave functions as neural networks but instead directly model the generative process that generates electron coordinates. This enables the novel generative Schrödinger network 800 to estimate the integral using the random samples from the generative networks in parallel. Thus, the generative Schrödinger network 800 is more efficient than known approaches (e.g., neural networks embodied in VMC, MCMC, and the like) to applying AI techniques to solve the Schrödinger equation (e.g., through Equation-1 and Equation-7 shown in FIG. 3A and FIG. 3B, respectively). These known MCMC-based approaches use sequential random sampling methods, which are slower and harder to scale than the parallel sampling methods used in the generative Schrödinger network 800.

As shown in FIG. 8, the generative Schrödinger network 800 includes a uniform samples module 810, a prior model 820, a decoder model 830, a generative model 840, a random coordinate samples module 850, an anti-symmetry correction model 860, an anti-symmetry constraint module 870, and a loss minimization module 880, configured and arranged as shown. The uniform samples module 810 is coupled in parallel to the prior model 820 and the decoder model 830. The prior model 820 and the decoder model 830 each provide inputs, in parallel, to the generative model 840, and the generative model 840 is randomly sampled by the random coordinate samples module 850. The random coordinate samples module 850 provide inputs to the anti-symmetry correction model 860 and the anti-symmetry constraint module 870, and the anti-symmetry constraint module 870 receives inputs from both the random coordinate samples module 850 and the anti-symmetry correction model 860. The anti-symmetry constraint module 870 provides input the loss minimization module 880.

The generative model 840 can be implemented using any suitable generative network. In general, generative networks utilize generative modeling, which is a type of unsupervised learning problem that automatically discovers and learns the regularities or patterns in input data in such a way that the model can be used to generate or output new examples that plausibly could have been drawn from the original dataset. Examples of suitable generative algorithms that can be used to implement aspects of the invention include generative adversarial networks (GANs) and auto-encoders (AEs) (e.g., a variational AE (VAE)).

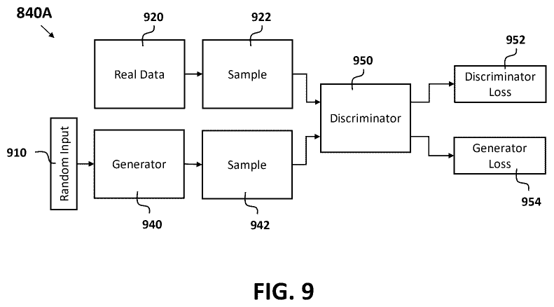

FIG. 9 depicts a non-limiting example of how the generative model 840 (shown in FIG. 8) can be implemented as generative adversarial network (GAN) 840A. The GAN 840A can model the distribution of data by imitating that distribution. For example, the generator can model a distribution by producing convincing “fake” data that looks like it's drawn from that distribution. The GAN 840A pairs a generator 940, which learns to produce the target output, with a discriminator 950, which learns to distinguish true data from the output of the generator 940. The generator 940 tries to fool the discriminator 950, and the discriminator 950 tries to keep from being fooled. More specifically, the generator 940 learns to generate plausible data (e.g., samples 942), and the generated instances (e.g., samples 942) from the generator 940 become negative training examples for the discriminator 950. The discriminator 950 learns to distinguish the generated instances (e.g., samples 942) from the generator 940 from real data (e.g., samples 922 of the real data 920). The discriminator 950 penalizes the generator 940 for producing implausible results. When training of the GAN 804A begins, the generator 940 produces obviously fake data, and the discriminator 950 quickly learns to tell that it's fake. As training continues, the generator 940 moves closer to producing output that can fool the discriminator 950. If training proceeds well, the discriminator 950 becomes less able to tell the difference between fake and real data and will begins classifying fake data as real. The generator 940 and the discriminator 950 can be implemented as neural networks. The samples 942 output from the generator 940 are fed directly to the discriminator 950.

The discriminator 950 performs classification operations to output classifications of the samples 922, 942 as real or fake, along with a discriminator loss 952 and a generator loss 954. Through backpropagation, the discriminator 950 uses the discriminator classification outputs and the discriminator loss 952 to train the discriminator 950 without training the generator 940. During training of the discriminator 950, the discriminator 950 ignores the generator loss 954 and just uses the discriminator loss 952. The discriminator loss 952 penalizes the discriminator 950 for misclassifying an instance of the real sample 922 as fake or for misclassifying an instance of the fake sample 942 as real. The discriminator 950 updates its weights through backpropagation from the discriminator loss 952. generator 940 does not train. and discrimination provides a signal that the generator uses to update its weights.

During training of the generator 940, the generator 940 learns to create fake data by incorporating feedback from the discriminator 950. More specifically, the generator 940 learns to make the discriminator 950 classify its output samples 942 as real. Training the generator 940 also uses backpropagation but requires tighter integration between the generator 940 and the discriminator 950 than discriminator training requires. The portion of the GAN 840A that trains the generator 940 includes the random input 910, the generator 940, the discriminator 950, the classifications generated by the discriminator 950, and the generator loss 954. The random input 910 can begin as random noise that is that the generator 940 will over time transform into meaningful outputs. The backpropagation used during training of the generator 940 flows from the generator loss 954 back through the discriminator 950 into the generator 940 to obtain gradients, which are used to change only the weights of the generator 940. The training process for the overall GAN 840A alternates between the discriminator 950 training for one or more epochs; and the generator 940 training for one or more epochs until the GAN 840A converges.

Returning now to FIG. 8, an overview of the generative Schrödinger network 800 will now be provided. The prior model 820 takes a random latent variable z sampled uniformly from the interval [0, 1] in uniform samples module 810 and produces a scalar value. The prior model 820 can be implemented as a simple neural network with linear layers and ReLU activation. With a set of latent variables {z1, z2, . . . , zL} assembled, a Softmax layer can be used to produce the probability distribution πj (line 3 in Algorithm-1 shown in FIG. 11).

The decoder model 830 is similar to the prior model 820 with linear layers and ReLU activation. The decoder model 830 produces the mean and the covariance matrix of the Gaussian distribution corresponding to the input latent z (lines 4-5 in Algorithm-1 shown in FIG. 11). The Generative model 840 combines the outputs from the prior model 820 and the decoder model 830 to create a mixture of L Gaussian components (line 6 in Algorithm-1) Ψ²(R) from which we can draw random samples S={R₁, R₂, . . . , R_{K}} (line 7 in Algorithm-1). The anti-symmetry correction model 860 can be implemented as a neural network taking three-dimensional coordinates of an electron and predicting a scalar value. Similar to the prior model 820 and the decoder model 830, alternating linear and ReLU layers are used to represent χ(β, r) (line 8, Algorithm-1) in the anti-symmetry correction model 860. Finally, the generative model Ψ(R) 840 and the anti-symmetry correction function C(R) are combined to create the anti-symmetry constraint loss (line 9 in Algorithm-1) and the estimate of the energy (line 10 in Algorithm-1), which results in the overall loss function (line 11 in Algorithm-1). The model parameters (θ, λ, β) are updated using the gradient of the loss functions in the descent direction. These steps are repeated until convergence.

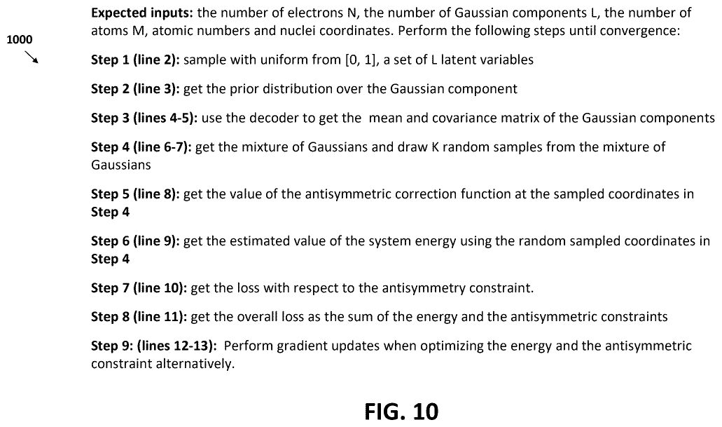

FIG. 10 illustrates a computer-implemented methodology 1000 that can be implemented by the generative Schrödinger network 800 (shown in FIG. 8) and/or Algorithm-1 (i.e., the SchrödingerNet Algorithm) (shown in FIG. 11). The following description references relevant steps of the methodology 1000, relevant components of the generative Schrödinger network 800, and relevant lines of the Algorithm-1. Expected inputs to the methodology 1000 and the generative Schrödinger network 800 (e.g., the uniform samples module 810) are taken from a SUE (e.g., the SUE 400 shown in FIGS. 4B, 5B, 6B), which includes the number of electrons N, the number of Gaussian components L, the number of atoms M, atomic numbers and nuclei coordinates. The steps of the methodology 1000 are performed until convergence. At Step 1 (line 2 of Algorithm-1), the prior model 820 and the decoder model 830 sample, in parallel, from a set of L latent variables of the unformed samples module 810 samples with uniformity from [0, 1]. At Step 2 (line 3 of Algorithm-1), use the prior model 820 to obtain a “prior” distribution over a Gaussian component. At Step 3 (lines 4-5 of Algorithm-1), use the decoder 830 to get the mean and covariance matrix of the Gaussian components. At Step 4 (line 6-7 of Algorithm-1), the generative model 840 is used to, based on outputs from the prior model 820 and the decoder model 830, generate a mixture of Gaussian components. The random coordinate samples module 850 is used to draw K random samples from the mixture of Gaussian components in the generative model 850. At Step 5 (line 8 of Algorithm-1), the anti-symmetry correction model 860 is used to get the value of the anti-symmetric correction function at the sampled coordinates in Step 4. At Step 6 (line 9 of Algorithm-1), use the anti-symmetry constraint module 870 to get the estimated value of the system energy using the random sampled coordinates in Step 4. At Step 7 (line 10 of Algorithm-1), use the anti-symmetry constraint module 870 to get the loss with respect to the anti-symmetry constraint. At Step 8 (line 11 of the Algorithm-1), use the loss minimization module 880 to get the overall loss as the sum of the energy and the antisymmetric constraints. At Step 9 (lines 12-13 of Algorithm-1), use the loss minimization module 880 to perform gradient updates when optimizing the energy and the antisymmetric constraint alternatively.

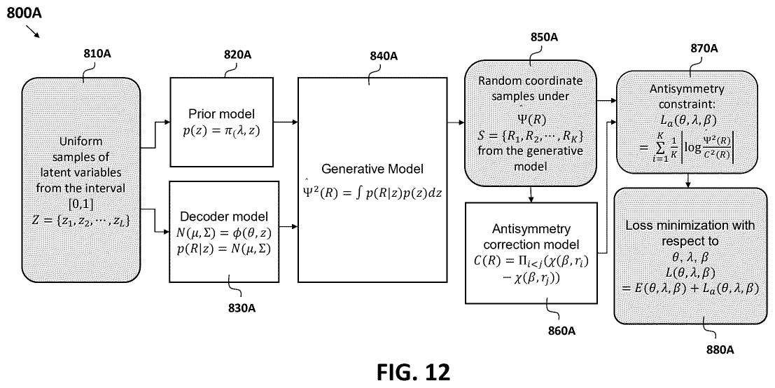

FIG. 12 depicts a non-limiting example of how the generative Schrödinger network 800 (shown in FIG. 8) can be implemented as a generative Schrödinger network 800A that includes a uniform samples module 810A, a prior model 820A, a decoder model 830A, a generative model 840A, a random coordinate samples module 850A, an anti-symmetry correction model 860A, an anti-symmetry constraint module 870A, and a loss minimization module 880A, configured and arranged as shown. The uniform samples module 810A is coupled in parallel to the prior model 820A and the decoder model 830A. The prior model 820A and the decoder model 830A each provide inputs, in parallel, to the generative model 840A, and the generative model 840A is randomly sampled by the random coordinate samples module 850A. The random coordinate samples module 850A provide inputs to the anti-symmetry correction model 860A and the anti-symmetry constraint module 870A, and the anti-symmetry constraint module 870A receives inputs from both the random coordinate samples module 850A and the ant-symmetry correction model 860A. The anti-symmetry constraint module 870A provides input the loss minimization module 880A.

A non-limiting example operation of the generative Schrödinger network 800A will now be provided. In accordance with aspects of the invention, the operations of the generative Schrödinger network 800A also track the operations performed using Algorithm-1 shown in FIG. 11. The operations begin by approximating the wave-function with a density \hat{Ψ}²(R) that depends on generative model(s) (e.g., 820A, 830A, 840A) using the equation \hat{Ψ}²(R)=p(R)=∫p(R|z)p(z)dz, where p(R|z) is modeled with a generative model parameterized by trainable parameters θ that generates R from the latent variable z. This generative model is implemented as the decoder model 830A. The distribution p(z) is a prior distribution of the latent variable with the parameter λ modeled by a generative model (e.g., the prior model 820A) that takes a random sample from a uniform distribution over the interval [0,1] and generates the latent variable z. Following the variational auto-encoder method, the decoder model 830A models p(R|z) as a multi-variate Gaussian distribution of N three-dimensional variables R=(r₁, r₂, . . . , r_{N}). In particular, the parameters of a multivariate Gaussian distribution are denoted as (μ, Σ), where μ is the mean vector of size 3N and Σ is the covariance matrix of size 3N×3N. The portion of the decoder model 830A that generates the Gaussian parameters is denoted as ϕ(θ, z), i.e. a neural network that decodes a latent variable z to predict the mean and the covariance matrix of the Gaussian distribution that generates the random sample R. Returning to the decoder model 830A, the probability density function p(R|z) can be represented as shown by Equation-9 (shown in FIG. 3C), where Σ⁻¹ and |Σ| denote the inverse and the determinant of the covariance matrix, respectively.

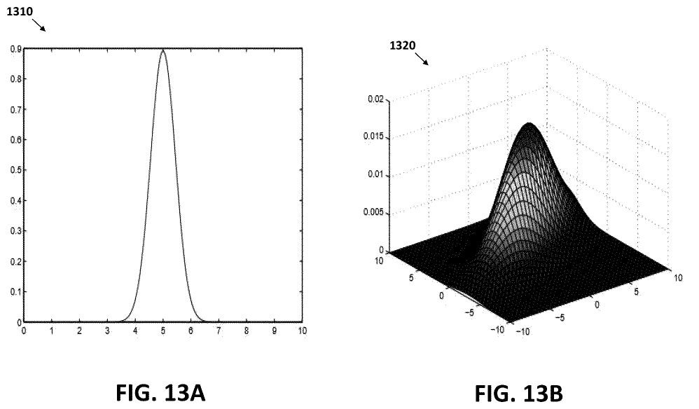

Additional details of Gaussian distributions used in accordance with embodiments of the invention are shown in FIGS. 13A and 13B. FIG. 13A depicts a univariate Gaussian distribution 1310; and FIG. 13B depicts a multivariate Gaussian distribution 1320 in accordance with aspects of the invention. The multivariate Gaussian distribution 1320 is bivariate for ease of illustration. However, in accordance with aspects of the invention, the multivariate Gaussian distribution 1320 can include higher dimensions (more than two (2) variables). In general, a multivariate is a vector with each of its elements being a variate. The variates need not be independent, and if they are not, a correlation is said to exist between them. The term “multivariate” is also used as an adjective to mean involving many variables, as opposed to one (univariate) or two (bivariate). In comparison to the multivariate Gaussian distribution 1320, the one-dimensional Gaussian distribution 1310 has a two-dimensional (2D) bell shape. The “central limit theorem” demonstrates that sums of large numbers of independent, identically distributed random variables are well approximated by a Gaussian distribution. The parameter estimates in a statistical model are also asymptotically Gaussian. Embodiments of the invention utilize Gaussians in probabilistic modeling for these reasons, together with the fact that Gaussian distributions can be efficiently manipulated using the techniques of linear algebra. The parameters of an n-dimension multivariate Gaussian distribution are an n-dimensional mean vector and an n-by-n dimensional covariance matrix. In other words, the multivariate Gaussian distribution 1320 represents the distribution of a multivariate that is made up of multiple random variables that can be correlated with each other. In probability, and in statistics, a multivariate random variable or random vector is a list of mathematical variables each of whose value is unknown, either because the value has not yet occurred or because there is imperfect knowledge of its value. The multivariate Gaussian distribution 1320 is defined by sets of parameters, namely the mean vector μ, which is the expected value of the distribution; and the covariance matrix Σ, which measures how dependent the random variables are and how they change together.

The prior model 820A utilizes a prior p(z), which can be parameterized by a neural network using Equation-10 (shown in FIG. 3C), where λ is the trainable parameter of the network π(λ, z) and c=∫π(λ, z)dz is a normalization factor because π(λ, z) is not necessarily a proper probability distribution function. Using the given parameterization, the wave function can be written as Equation 11 (shown in FIG. 3C), which can be approximated as Equation 12 (shown in FIG. 3C), where

$${π}_{j} = \frac{π ( λ , {z}_{j} )}{{\sum }_{j = 1}^{L} π ( λ , {z}_{j} )} ,$$

and {z₁, z₂, . . . , z_{L}} is a random samples of latent variables from the interval [0,1]. It should be noted that

$${\sum }_{j = 1}^{L} {π}_{j} = 1.$$

Thus, the approximation of the wave function used herein has a form that is a mixture of Gaussian components. This formulation does not have a fixed mixture of Gaussian components but it is dynamic in the sense that the mixture of Gaussian components is dependent on the sampled latent variables.

The generative Schrödinger network 800A also takes into account anti-symmetry considerations. Because electrons are fermions, they must follow the Pauli-exclusion principle, i.e., the wave functions of same-spin electrons must be anti-symmetric. Specifically, the sign of the function must be changed when two random coordinates of the same-spin electrons Ψ(r₁, . . . , rᵢ, . . . , rⱼ, . . . , r_{N})=−Ψ(r₁, . . . , rⱼ, . . . , rᵢ, . . . , r_{N}) are exchanged. The generative Schrödinger network 800A provides a method that regularizes the wave function such that it has a shape similar to an anti-symmetric function.

The generative Schrödinger network 800A provides anti-symmetry regularization through the random coordinate samples module 850A, the anti-symmetry correction model 860A, the anti-symmetry constraint module 870A, and the loss minimization module 880A, configured and arranged as shown. The asymmetric function shown in Equation-13 (shown in FIG. 3D) can be used, where rᵢ denotes the 3D coordinate of the ith electron and χ(β, rᵢ) is a neural network parameterized by β. It can be estimated that the given function is an anti-symmetric function. C(R) can be referred to as a correction function. Given a set of random samples S={R₁, R₂, . . . , R_{K}} from the distribution \hat{Ψ}²(R) we consider the additional constraint represented by Equation-14 (shown in FIG. 3D). The value of Lₐ(θ, λ, β) will be close to zero if and only if \hat{Ψ}²(R) is an approximation of C²(R). Adding up everything, the final loss function generated at the loss minimization module 880A is a combination of the system energy and the anti-symmetry regularization as shown in Equation-15 (shown in FIG. 3D) (L(θ, λ, β)=E(θ, Δ, β)+Lₐ(θ, λ, β)).

To validate the generative Schrödinger network 800A, standard systems with known ground-state energy were used. Those are small systems corresponding to one atom each with several electrons ranging from 1 to 10. The statistics about the systems are reported in Table 1400 shown in FIG. 14. Table 1500 shown in FIG. 15 shows total GPU time of different methods, including the SchrödingerNet (embodying aspects of the invention), PesNet, and FermiNet. PesNet and FermiNet rely on MCMC methods or alternative methods like diffusion Monte Carlo for estimating the integral involved in the energy calculation. Embodiments of the invention, on the other hand, models the generative process where the wave function is indirectly approximated via the density provided by the generative models. Therefore, embodiments of the invention can estimate the energy using random samples generated from the generative models. Because the generative models can generate samples in parallel, MCMC sequential sampling is not needed in embodiments of invention.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” are understood to include any integer number greater than or equal to one, i.e. one, two, three, four, etc. The terms “a plurality” are understood to include any integer number greater than or equal to two, i.e. two, three, four, five, etc. The term “connection” can include both an indirect “connection” and a direct “connection.”

The terms “about,” “substantially,” “approximately,” and variations thereof, are intended to include the degree of error associated with measurement of the particular quantity based upon the equipment available at the time of filing the application. For example. “about” can include a range of ±8% or 5%, or 2% of a given value.

What is claimed is:

1. A computer-implemented method comprising:
executing a generative network comprising a generative model of a system under evaluation (SUE);
wherein the generative model is operable to model a probability density function of the SUE to generate electron coordinates of electrons in the SUE;
wherein the electron coordinates are generated by the generative model in a manner that minimizes an estimated energy of the SUE; and
wherein the electron coordinates generated by the generative model can be sampled in parallel.

2. The computer-implemented method of claim 1, wherein the estimated energy of the SUE is associated with multiple energy sources.

3. The computer-implemented method of claim 2, wherein the multiple energy sources comprise a Laplacian energy source, an electron-nuclei energy source, and an electron-electron energy source.

4. The computer-implemented method of claim 1, wherein the probability density function comprises a multivariate Gaussian distribution.

5. The computer-implemented method of claim 1 further comprising using an anti-symmetry correction model to apply an anti-symmetry correction function to the probability density function generated by the generative model.

6. The computer-implemented method of claim 5 further comprising combining an output of the probability density function generated by the generative model and the anti-symmetry correction function.

7. The computer-implemented method of claim 6, wherein combining the output of the probability density function generated by the generative model and the anti-symmetry correction function creates an anti-symmetry constraint loss and an estimate of an energy of the SUE.

8. A computer-based system comprising:
a memory; and
a processor system communicatively coupled to the memory;
the processor system configured to perform processor system operations comprising executing a generative network comprising a generative model of a system under evaluation (SUE);
wherein the generative model is operable to model a probability density function of the SUE to generate electron coordinates of electrons in the SUE;
wherein the electron coordinates are generated by the generative model in a manner that minimizes an estimated energy of the SUE; and
wherein the electron coordinates generated by the generative model can be sampled in parallel.

9. The computer-based system of claim 8, wherein the estimated energy of the SUE is associated with multiple energy sources.

10. The computer-based system of claim 9, wherein the multiple energy sources comprise a Laplacian energy source, an electron-nuclei energy source, and an electron-electron energy source.

11. The computer-based system of claim 8, wherein the probability density function comprises a multivariate Gaussian distribution.

12. The computer-based system of claim 8, wherein the processor system operations further comprise using an anti-symmetry correction model to apply an anti-symmetry correction function to the probability density generated by the generative model.

13. The computer-based system of claim 12, wherein the processor system operations further comprise combining an output of the probability density function generated by the generative model and the anti-symmetry correction function.

14. The computer-based system of claim 13, wherein combining the output of the probability density function generated by the generative model and the anti-symmetry correction function creates an anti-symmetry constraint loss and an estimate of an energy of the SUE.

15. A computer program product comprising a computer readable program stored on a computer readable storage medium, wherein the computer readable program, when executed on a processor system, causes the processor system to perform processor system operations comprising:
executing a generative network comprising a generative model of a system under evaluation (SUE);
wherein the generative model is operable to model a probability density function of the SUE to generate electron coordinates of electrons in the SUE;
wherein the electron coordinates are generated by the generative model in a manner that minimizes an estimated energy of the SUE; and
wherein the electron coordinates generated by the generative model can be sampled in parallel.

16. The computer program product of claim 15, wherein the estimated energy of the SUE is associated with multiple energy sources.

17. The computer program product of claim 16, wherein the multiple energy sources comprise a Laplacian energy source, an electron-nuclei energy source, and an electron-electron energy source.

18. The computer program product of claim 15, wherein the probability density function comprises a multivariate Gaussian distribution.

19. The computer program product of claim 15 further comprising using an anti-symmetry correction model to apply an anti-symmetry correction function to the probability density generated by the generative model.

20. The computer program product of claim 19 further comprising:
combining an output of the probability density function generated by the generative model and the anti-symmetry correction function; and
wherein combining the output of the probability density function generated by the generative model and the anti-symmetry correction function creates an anti-symmetry constraint loss and an estimate of an energy of the SUE.
